(19) United States
(12) Patent Application Publication
(10) Pub. No.: US 2021/0098133 A1
(43) Pub. Date: Apr. 1, 2021

(54) Secure Scalable Real-Time Machine Learning Platform for Healthcare

(71) Applicant: Parkland Center for Clinical Innovation, Dallas, TX (US)

(72) Inventors: Vikas Chowdhry, Southlake, TX (US); Priyanka Kharat, Dallas, TX (US); Arun Nethi, Irving, TX (US); Akshay Arora, Irving, TX (US); Vency Varghese, Irving, TX (US); Steve Miff, Dallas, TX (US)

(21) Appl. No.: 17/033,667

(22) Filed: Sep. 25, 2020

Related U.S. Application Data
(60) Provisional application No. 62/907,539, filed on Sep. 27, 2019.

(51) Int. Cl. G16H 50/30; G06N 20/00; G16H 10/60; G06N 5/02

(52) U.S. Cl. CPC G16H 50/30; G06N 5/02; G16H 10/60; G06N 20/00

(57) ABSTRACT

A machine learning system for healthcare applications comprises a data ingestion pipeline configured to automatically receive patient data including stored data from an EHR database and real-time data from a plurality of data sources, the data including, EHR records, claims data, and social determinants of health data; a data processing module configured to clean, extract, and process the received patient data; at least one predictive model configured to analyze the cleaned and processed data and determine a risk score for each patient; a configuration file defining the predictive model execution parameters; a tuning module configured to adjust parameters of the predictive model, including variables, thresholds, and coefficients; a retraining module configured to make further adjustments of the predictive model to remove inherent data biases; and a dashboard and reporting module configured to present the risk score to a patient care team.

### RELATED APPLICATION

This application claims the benefit of U.S. Provisional Application No. 62/907,539 filed Sep. 27, 2019, which is incorporated herein by reference in its entirety.

### FIELD

The present disclosure relates generally to a computing platform, and in particular to a secure real-time machine learning platform in the field of disease identification, patient care, and patient monitoring that facilitates predictive model development, deployment, evaluation, and retraining.

### BACKGROUND

In recent times, Machine learning (ML) based systems have evolved and scaled across different industries such as finance, retail, insurance energy utilities etc. Among other things, they have been used to predict patterns of customer behavior, to generate pricing models and to predict return on investments. But the successes in deploying machine learning models at scale in those industries has not translated into healthcare setting.

### BRIEF DESCRIPTION OF THE DRAWINGS

FIG. 1 is a simplified block diagram of the exemplary system and method for predictive model development, deployment, evaluation, and retraining using machine learning according to the teachings of the present disclosure;

FIG. 2 is a simplified block diagram of an exemplary embodiment of an end-to-end cloud hosted machine learning platform according to the present disclosure;

FIG. 3 is a simplified block diagram of an exemplary embodiment of a data orchestration engine according to the present disclosure;

FIG. 4 is a simplified block diagram of an exemplary embodiment of a configuration-based workflow according to the present disclosure;

FIG. 4 is a simplified block diagram of an exemplary embodiment of a disaster recovery and fault tolerance architecture according to the present disclosure; and

FIG. 5 is a simplified block diagram of an exemplary embodiment of machine learning workflow with clinical decision support according to the present disclosure.

### DETAILED DESCRIPTION

The present disclosure describes a machine learning (ML) framework/platform/system to seamlessly develop, test, deploy, evaluate and retrain predictive models by reducing the time to market for integrating clinical and environmental predictive insights in healthcare workflows to make them actionable. Part of the motivation to build such a flexible but scalable and configurable framework is due to the curated set of data transformation techniques that data scientists perform in terms of imputation, categorical encoding of continuous variables or aggregation of healthcare datasets before using them to train a predictive model in the development flow.

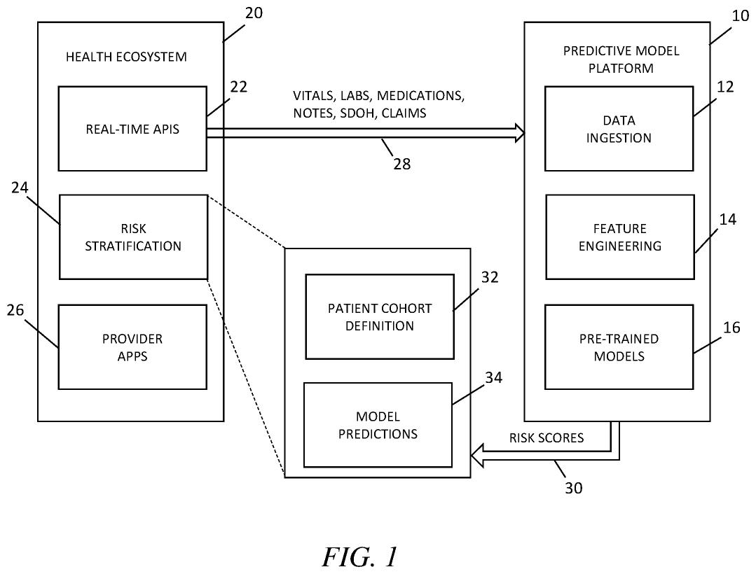

FIG. 1 is a simplified block diagram of the exemplary system and method 10 that are configured for predictive model development, deployment, evaluation, and retraining using machine learning according to the teachings of the present disclosure. As shown in FIG. 1, the system and method 10 are shown in deployment mode, which include a data ingestion component 12, feature engineering component 14, and pre-trained predictive models 16. The system and method 10 execute code that receives and processes the data received from a plurality of sources 18 originating from or are associated with a healthcare system 20, via real-time APIs (Application Program Interfaces) 22 that provide a channel for bidirectional real-time data 28, such as patients' vitals, lab results, medications, physicians' and nurses' notes, Social Determinants of Health (SDOH) data, and claims data to the system and method 10. It is contemplated that the system and method 10 may also ingest historical or non-real-time patient clinical and non-clinical data related to the patients for this process. Although the focus herein is in a healthcare setting, predictive models of various types may be developed, deployed, fine-tuned, and retrained using the system and method 10. Predictive models may include, for example, acute care models, chronic care models, operational return on the investment (ROI) models, and public health models. Users of the system and method 10 may include clinical care teams, data scientists, business analysts, machine learning engineers, and healthcare institution administrators.

Healthcare data by its very nature is highly complex, high dimensional, and of inconsistent quality. For this data to be useful, it needs a systematic data ingestion approach to collect, store, and integrate data-driven insights into their clinical and operational processes. To quickly ingest this multi-dimensional data and scale, a configurable and flexible data ingestion pipeline solution is used to ingest all the relevant health data such as clinical data (e.g., electronic health record or EHR), claims data, Social Determinants of Health, and streaming Internet of things (IoT) data. The data ingestion pipeline may also ingest genomics data and high-quality diagnostic imaging data. The platform may ingest, for example, sensor data from indoor air quality IoT sensors via the ingestion pipeline API. The ingested data is then cleaned in batch mode using the data cleaning modules in the platform. The IoT data is stored and maintained in a database on the platform with fault tolerance and disaster recovery functionalities. The IoT data may be integrated with the existing machine learning models to add more features that further improve the predictive model performance.

The electronic medical record (EMR) clinical data may be received from entities such as hospitals, clinics, pharmacies, laboratories, and health information exchanges, including: vital signs and other physiological data; data associated with comprehensive or focused history and physical exams by a physician, nurse, or allied health professional; medical history; prior allergy and adverse medical reactions; family medical history; prior surgical history; emergency room records; medication administration records; culture results; dictated clinical notes and records; gynecological and obstetric history; mental status examination; vaccination records; radiological imaging exams; invasive visualization procedures; psychiatric treatment history; prior histological specimens; laboratory data; genetic information; physician's notes; networked devices and monitors (such as blood pressure devices and glucose meters); pharmaceutical and supplement intake information; and focused genotype testing. The EMR non-clinical data may include, for example, social, behavioral, lifestyle, and economic data; type and nature of employment; job history; medical insurance information; hospital utilization patterns; exercise information; addictive substance use; occupational chemical exposure; frequency of physician or health system contact; location and frequency of habitation changes; predictive screening health questionnaires such as the patient health questionnaire (PHQ); personality tests; census and demographic data; neighborhood environments; diet; gender; marital status; education; proximity and number of family or care-giving assistants; address; housing status; social media data; and educational level. The non-clinical patient data may further include data entered by the patients, such as data entered or uploaded to a patient portal. Additional sources or devices of EMR data may provide, for example, lab results, medication assignments and changes, EKG results, radiology notes, daily weight readings, and daily blood sugar testing results. Additional non-clinical patient data may include, for example, gender; marital status; education; community and religious organizational involvement; proximity and number of family or care-giving assistants; address; census tract location and census reported socioeconomic data for the tract; housing status; number of housing address changes; frequency of housing address changes; requirements for governmental living assistance; ability to make and keep medical appointments; independence on activities of daily living; hours of seeking medical assistance; location of seeking medical services; sensory impairments; cognitive impairments; mobility impairments; educational level; employment; and economic status in absolute and relative terms to the local and national distributions of income; climate data; health registries; the number of family members; relationship status; individuals who might help care for a patient; and health and lifestyle preferences that could influence health outcomes. Certain data identified above are referred to as social determinants of health (SDOH) data that provide insight into the conditions in which people are born, grow, live, work and age, and may include factors like socioeconomic status, education, neighborhood and physical environment, employment, and social support networks, as well as ease of access to health care.

Certain selected data dependent on the model being deployed are processed using feature engineering methods to extract meaning and generate binary values (yes or no) from the data. For example, a patient data involving one or more variable values, such as blood glucose, is interpreted as positive for diabetes, when that value exceeds a predetermined threshold. Another example is the translation of certain diagnostic codes to a binary value (yes or no) for certain health conditions. Additionally, patient data such as physicians' and nurses' notes are processed using natural language processing (NLP) methods to extract useful meaning or interpretation. The ingested and processed data then serve as input to one or more predictive models that have been pre-trained (or verified as being accurate). Each predictive model provides an assessment of each patient's risk for a certain health condition. The result is one or more risk scores 30 for each patient that provide insight on whether the patient is likely to contract a certain disease or encounter a certain adverse event.

The computed risk scores 30 are presented on specialized dashboards and reports to the healthcare team that enables the team members to define patient cohort 32 and model predictions 34 and stratify the patients stratified by risk 24. For example, the dashboard and/or report may identify those patients who are at the highest risk for developing sepsis and therefore should receive focused immediate attention, patients who are at medium risk for developing sepsis, and patients who are not at risk for developing sepsis. The healthcare system 20 may additionally deploy certain provider applications 26 that enable the healthcare team to further utilize the risk scores and derive functionality.

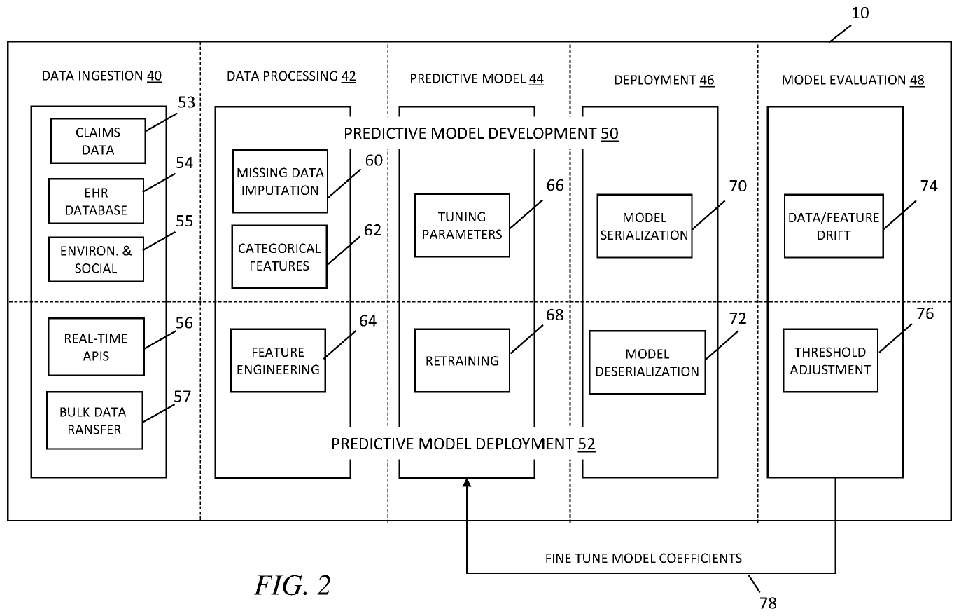

As shown in FIG. 2, the system and method 10 includes five primary functions: data ingestion 40, data processing 42, predictive models 44, model deployment 46, and model evaluation 48 that are used in the model development 50 and model deployment 52 workflows. As described above, data ingestion 40 is the process in which historical and real-time clinical and non-clinical data related to the patients are accessed and received. These data sources are stored in claims database 53, EHR database 54, and environmental & social database 55, or are ingested via APIs 56 for real-time data or using bulk data transfer mechanisms 57. Part of the data processing function 42 for predictive model development 50 is determining data that are missing or not available 60. This function may look at past data points to extrapolate the values of missing data points. This function may also impute past missing data points using current data points. Data processing 42 also includes assigning a category 62 to certain patient data parameters by comparing the parameter values to a threshold or a range of values. For example, a patient's blood glucose may be assigned to the “bad” category because it exceeds a certain threshold or falls within a certain range. Categorical data are variables that contain label values rather than numeric values.

As a part of predictive model development 50, the parameters of the predictive model 44 are fine-tuned 66 to increase the accuracy of the model. Predictive model serialization 70 is a way to efficiently express a predictive model in the system so it can be run in real-time during deployment 46 using real-time patient data. The predictive model may be evaluated 48 by detecting and correcting for data/feature drift 74 that may occur over time. Data/drift detection can be done by monitoring the performance of the predictive model to actual data.

As part of predictive model deployment 52, data processing 42 also includes feature engineering 64, which converts input data to a binary value that is indicative of the patient's condition, such as whether the patient has diabetes etc. One-hot encoding is a type of feature engineering. As part of deployment, the predictive model 44 undergoes retraining 68 using actual real-time data. During deployment 46, the serialized predictive model undergoes deserialization 72 so that it can be “executed.” As part of the model evaluation 48, the thresholds of the predictive model are adjusted 76 to correct for inaccuracies, and fine-tune coefficients 78 are generated and used for retraining the predictive model. The platform allows retraining of the predictive models using the same data set that was ingested into the model through APIs. The platform leverages this data set and generates multiple versions of the predictive model by simply editing the model signature. The platform facilitates the data scientists to perform statistical tests to keep the predictive models updated with new incoming data streams.

Therefore in this manner, there is consistency in the way features are created for model training and model scoring. Thus, there is standardization of training and deployment/scoring workflow which further helps in quickly learning through prospective testing of the key components, which can trigger data or feature drift as the model runs in real environment. This is done in the same controlled environment that can ingest either historical or real-time data through the same APIs or secure connections. To achieve this, the entire framework is hosted in a secure HIPAA-compliant cloud infrastructure to deploy as a turn-key solution.

This system is hosted on cloud-based infrastructure such as Microsoft Azure Cloud Platform, which enables state-of-the-art functionalities like network security, data replication, disaster recovery and fault tolerance needed for any robust and enterprise-grade software-as-a-service (SaaS). Cloud resources (compute and storage) leverage economies of scale to keep cost to a realistic level without having a needed to maintain a large healthcare information technology (HIT) professional staff. Thus, being cost-effective as well as scalable and configurable, this system can be adopted by health organizations of a wide range of sizes.

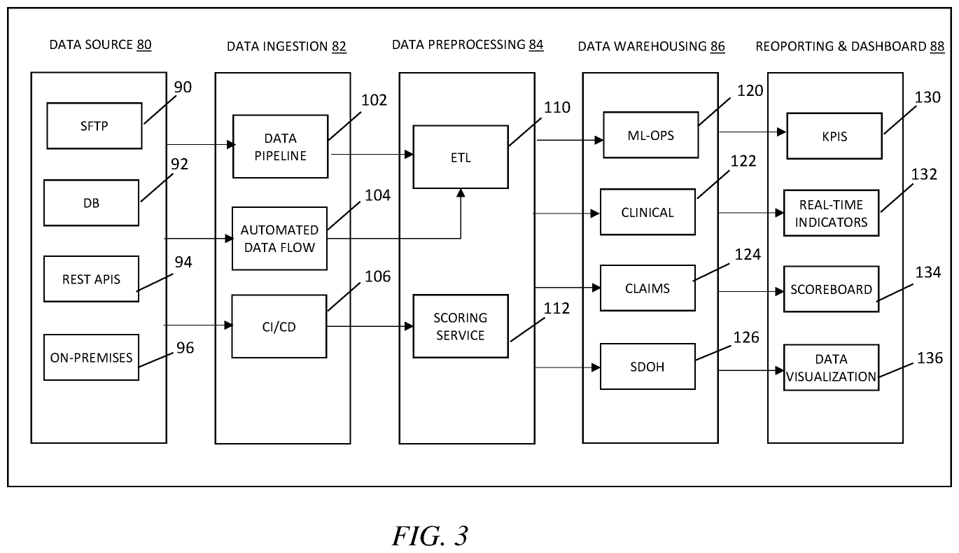

Referring to FIG. 3, the data ingestion pipeline 82 from the data sources 80 is based on an architecture that enables user-defined transformations for real-time data scoring, cleaning, and de-duplication without requiring additional middleware. The data sources include data accessed by Secure File Transfer Protocol (SFTP) 90 and from databases 92. Raw data may also be obtained by making RESTful API calls 94 to the EHR API servers or through regular intervals of data fetch using secure file transfer process. Generally, these API servers are the hub for all the API requests that facilitates the connection between the EHR organizational users and the operational database management system to stream near real-time data seamlessly as a json response through the web service APIs upon service requests. Additional data sources may be data that are generated and/or stored on-premises 96. Therefore, the data ingestion process 82 includes a data pipeline 102, an automated data flow 104, and a continuous integration/continuous deployment/delivery (CI/CD) 106. The data pipeline is fully automated, and it ingests the patient data in batch mode, where the batch size is based on the Service Level Agreement (SLA) requirements. Thus, the pipeline may be scheduled to trigger based on the SLA requirements and it continuously pulls the data from the APIs and performs the desired transformation and filtering operations. The concept of CI/CD 106 focuses on ongoing automation and continuous monitoring throughout the lifecycle of the software, from integration and testing phases to delivery and deployment.

Continuing to refer to FIG. 3, data preprocessing 84 includes extract, transform, and load (ETL) 110 the patient data from the data pipeline 102 and automated data flow 104. Data extraction involves extracting data from homogeneous or heterogeneous sources; data transformation processes data by data cleaning and transforming them into a proper storage format/structure for the purposes of querying and analysis; finally, data loading describes the insertion of data into the final target database such as an operational data store, a data mart, data lake or a data warehouse. The patient-level raw json data is preprocessed using an imputation and filter logic which transforms this data into clinically relevant features and are fed to the machine learning models using a scoring logic script to predict the risk of the acute care condition or other health condition risks based on the pre-trained model. The data imputation logic is used to fill in missing data so that predictions are realistic and accurate. Data preprocessing 84 also includes scoring services 112 that involve deploying the predictive model(s) to generate risk scores using data from CI/CD 106. The scoring script generates the score response which encompasses the transformed features and the identified risk levels associated with the patient. These responses are aggregated in batch mode and after cleaning they are converted into SQL tables using a database operation script and ingress into the Postgres SQL database, where this data is stored in a secure and reliable manner. The raw json responses are pushed to a data repository such as Azure Data Lake to preserve the raw patient-level information for audit purposes.

Data warehousing 86 includes storing the risk scores, machine learning operations (ML-OPS) 120, clinical data 122, claims data 124, and social determinants of health data 126. The warehoused data are securely stored with backups. The healthcare team members may access the warehoused data by viewing subsets of the data presented in a variety of ways on the screen and in report form, including key performance indicators (KPIs) 130, real-time indicators 132, scoreboard 134, and data visualization 136 methods. This may include enabling the user to view the data according to certain key performance indicators (KPIs) 130. For example, a user may ask the system to determine what percentage of the patient population are at risk for sepsis. Further, historical data sets may be accessed while the predictive model is running live in production. These data sets are pushed to a model explainer script that extracts the top contributing features that helped to arrive at the risk score predictions. This feature is especially useful to clinicians for making real-time decisions.

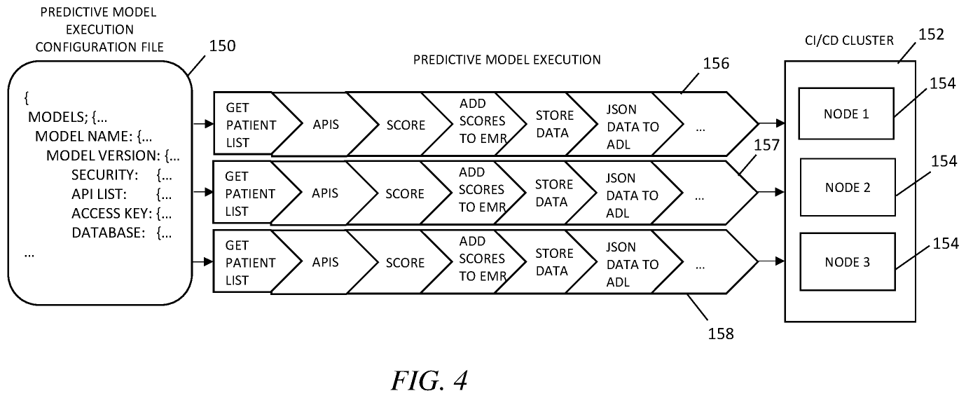

The platform provides a unique way of deploying and executing predictive model workflow for scoring using a single codebase that can support multiple models and versions. using a configuration file 150 as shown in FIG. 4. The configuration file contains information about how the predictive model should be run, including name of the model, version, security, API, access key, database, location of the model, frequency to run the model, etc. It is also designed to use a single infrastructure cluster 152 containing multiple computing nodes 154 to execute any number of scoring workflow pipelines 156-158 parallel and automate the scoring process using continue integration and continuous deployment/delivery process (CI/CD). The use of the configuration file methodology facilitates easy upgrade to an existing model or serving a new model in the pipeline workflow as it has a very short delivery cycle.

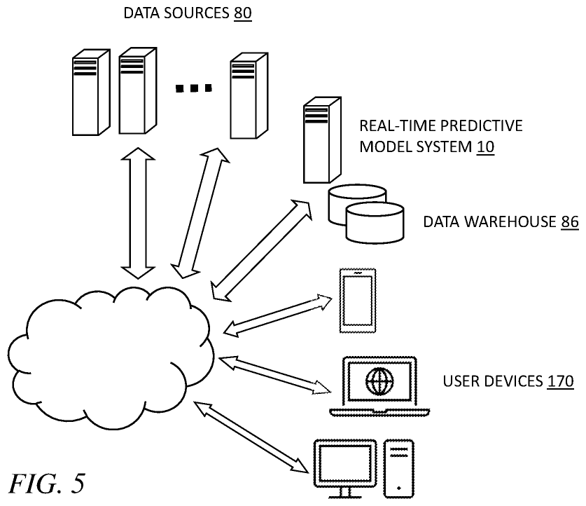

FIG. 5 is a simplified block diagram for the operational environment of the system and method described herein. The real-time system and method 10 can be hosted on a cloud-based platform (e.g., Azure) with cloud-based data warehouses 86 that are configured to access and receive patient clinical and non-clinical data sources 80 via data pipeline, automated data flow, and real-time API as described above. Users may access the reporting and dashboard functions of the system and method 10 via a variety of computing devices 170, including, for example, mobile devices, laptop computers, notebook computers, notepads, and desktop computers. The cloud-based solution facilitates data replication, fault tolerance, and computational and data scalability without an on-premises infrastructure with enormous upfront investment. Further, load-balancing and database redundancy and mirroring mechanisms may be deployed to implement a fault-tolerant system.

The cloud-based platform may leverage cloud-based security policies such as the Azure active directory-based service for access control to manage applications and hosted services on the cloud and handle sensitive information (PHI). This eliminates the need for user-level login to the cloud applications. Azure RBAC uses Active Directory policies for managing the authentication. This platform provides a single role-based access to multi-institutional EHR data. Additionally, this platform also provides a comprehensive, immutable log management service with easy access across deployed applications using elastic search and the Kibana dashboard, which ensures a single point of reference to test for any application-level logs or system-level logs in a responsible manner. Using app-insight notifications, the platform provides real-time alerts for any configured event like an exception in application or missing data from the source API.

The system is engineered to overcome these shortcomings and has the capabilities to scale up and accelerate the prediction model workloads to meet the needs of high-performance computing, low-latency, high-bandwidth network communication, memory-intensive requirements. This cloud-based solution resolves problems such as infrastructure upgrade, scalability, transfer and deployment at multiple locations using automated process and containerization. This has considerably reduced the cost of infrastructure and engendered flexibility for migration/deployment on the cloud environments with minimal application-level changes for the code, database, and the data model architecture.

The system includes well-defined replication graphs and disaster recovery strategies for their database and support systems by imposing identical servers running in parallel replication with a mirrored backup of database and system-level logs to ensure high levels of data availability. These applications are designed using the microservices-based architecture to reduce the redundancies from all the key components by performing similar activities in each workflow.

The system and method 10 further including a logging service that records logging information in real-time that can help to validate the stability of the system through warning and debug logs. This log data is fed to a high scale analytical engine (elastic search) which enables full-text searches and can be integrated with a visualization dashboard like Kibana to provide feeds to self-hosted web-front application using restful APIs. This visualization provides monitors and performance metrics based on application-level logs of the automated pipeline for predictive and analytical applications. This also ensures quality delivery of the model serving on this platform and a quick debugging capability for any production outage.

For any production environment that is automated, having a notification system is critical given the fact that no workflow/infrastructure is perfect. In addition to the log management system, a slack based notification service is also integrated with the platform to generate real-time alerts about the production pipeline so that the engineering and data science teams may be fully aware of the live status of the pipeline and the patient risk scores. The notification system captures both infrastructure and application failures/exceptions. Thus, this alerting system ensures immediate action and remediation in case of any failed events

The platform is designed to be a generic multipurpose data science engine. The flexible architecture of this platform allows the use of functional decision-making modules that can run asynchronously without disrupting the integrity of the system. The prediction service on the platform can be leveraged by the model evaluation service where real-time predictions can be interpreted by the models on the fly thereby making it extremely useful for the data scientists and clinicians (or stakeholders) to get actionable insights.

The platform is an end-to-end system for developing and deploying machine learning models. Using this platform, data scientists can use machine learning toolkits and libraries to create models, perform statistical tests and deploy them. The platform architecture supports the sharing of pretrained models across different ML module run-time environments. As illustrated by the case studies, the platform provides project-level isolation and code reusability, and demonstrates versatility in terms of providing a prediction service, IoT data ingestion, and SDOH integration.

The features of the present invention which are believed to be novel are set forth below with particularity in the appended claims. However, modifications, variations, and changes to the exemplary embodiments described above will be apparent to those skilled in the art, and the system and method described herein thus encompasses such modifications, variations, and changes and are not limited to the specific embodiments described herein.

What is claimed is:

1. A machine learning system for healthcare applications comprising:
a data ingestion pipeline configured to automatically receive patient data including stored data from an EHR database and real-time data from a plurality of data sources, the data including, EHR records, claims data, and social determinants of health data;
a data processing module configured to clean, extract, and process the received patient data;
at least one predictive model configured to analyze the cleaned and processed data and determine a risk score for each patient;
a configuration file defining the predictive model execution parameters;
a tuning module configured to adjust parameters of the predictive model, including variables, thresholds, and coefficients;
a retraining module configured to make further adjustments of the predictive model to remove inherent data biases; and
a dashboard and reporting module configured to present the risk score to a patient care team.

2. The system of claim 1, wherein the data ingestion pipeline comprises a plurality of application program interfaces configured to access real-time patient data.

3. The system of claim 1, wherein the data processing module comprises a missing data imputation module configured for determining values for missing patient data.

4. The system of claim 1, wherein the data processing module comprises a feature engineering module configured for determining a binary value for a data parameter in response to at least one value of at least one patient data parameter.

5. The system of claim 1, wherein the data processing module comprises a categorical feature module configured for determining a category for a data parameter in response to at least one value of at least one patient data parameter.

6. The system of claim 1, further comprising a model serialization module configured to express the predictive model in an efficient manner for storage.

7. The system of claim 6, further comprising a model deserialization module configured to convert the serialized model for execution.

8. The system of claim 1, further comprising a feature drift module configured to evaluate accuracy of the predictive model to detect drift.

9. The system of claim 1, further comprising a model threshold adjustment module configured to determine one or more model coefficients for fine-tuning the predictive model.

10. The system of claim 1, wherein the dashboard and reporting module is configured to present patients classified by their risk scores.

11. The system of claim 1, wherein the dashboard and reporting module is configured to present at least one patient data parameter that is a top contributor to a high risk score.

12. The system of claim 1, wherein the configuration file specifies a name, version, data source, data warehouse, execution frequency related to the execution of at least one predictive model.

13. The system of claim 1, further comprising a data warehousing module configured to store the risk score as a part of the patient's electronic medical record.

14. The system of claim 1, where the data ingestion pipeline is configured to ingest sensor data from at least one IoT sensor.

15. A predictive model method for healthcare applications comprising:
automatically ingesting patient data including stored data from an EHR database and real-time data from a plurality of data sources, the data including, EHR records, claims data, and social determinants of health data;
automatically cleaning, extracting, and processing the ingested patient data;
analyzing the cleaned and processed patient data using at least one predictive model and determining at least one risk score for each patient;
automatically sensing drift in the predictive model variables, thresholds, and coefficients;
automatically making adjustments of the predictive model to remove inherent data biases; and
presenting the at least one risk score to a patient care team.

16. The method of claim 15, further comprising executing the at least predictive model according to a configuration file defining the predictive model execution parameters.

17. The method of claim 15, wherein automatically ingesting patient data comprises ingesting real-time patient data via a plurality of application program interfaces.

18. The method of claim 15, wherein automatically processing the patient data comprises imputing values for missing patient data.

19. The method of claim 15, wherein automatically processing the data comprises determining a binary value for a data parameter in response to at least one value of at least one patient data parameter.

20. The method of claim 15, wherein automatically processing the data comprises determining a category for a data parameter in response to at least one value of at least one patient data parameter.

21. The method of claim 15, further comprising serializing the predictive model so that it is expressed in an efficient manner for storage.

22. The method of claim 21, further comprising deserializing the serialized model for execution.

23. The method of claim 15, further comprising evaluating the performance accuracy of the predictive model to detect drift.

24. The method of claim 15, further comprising determining one or more model coefficients for fine-tuning the predictive model.

25. The method of claim 15, wherein presenting the risk score comprises presenting the patients classified by their risk scores.

26. The method of claim 15, wherein presenting the risk score comprises presenting at least one patient data parameter that is a top contributor to a high risk score.

27. The method of claim 15, further comprising executing the at least one predictive model according to a configuration file that specifies a name, version, data source, data warehouse, execution frequency related to the execution of the at least one predictive model.

28. The method of claim 15, further comprising storing the at least one risk score as a part of the patient's electronic medical record.

29. The method of claim 15, wherein automatically ingesting patient data comprises ingesting sensor data from at least one IoT sensor.
